(19) United States
(12) Plant Patent Application Publication
(10) Pub. No.: US 2017/0118897 P1
(43) Pub. Date: Apr. 27, 2017

(54) HYDRANGEA PLANT NAMED 'SMNMAKTSR'

(50) ;

(71) Applicant: Timothy Wood, Spring Lake, MI (US)

(72) Inventor: Timothy Wood, Spring Lake, MI (US)

(21) Appl. No.: 14/756,864

(22) Filed: Oct. 22, 2015

(51) Int. Cl. A01H 5/00

(52) U.S. Cl. PLT/250

(57) ABSTRACT

A new and distinct Hydrangea cultivar named ‘SMNMAKTSR’ is disclosed, characterized by large lacecap inflorescence with single and double florets. Double florets are bright magenta/pink with yellow/green centers. Plants are cold hardy, with frost resistant flower buds and have a compact growth habit . The new variety is a Hydrangea, normally produced as an ornamental plant for containers or gardens.

Latin name of the genus and species: Hydrangea hybrid.

Variety denomination: ‘SMNMAKTSR’.

### BACKGROUND OF THE INVENTION

The new Hydrangea cultivar is a product of a planned breeding program conducted by the inventor, Timothy Wood, in Grand Haven, Mich. The objective of the breeding program was to produce hardy new Hydrangea varieties with a compact growth habit, reblooming plants with new flower types and colors. The open pollination resulting in this new variety was made during 2004.

The seed parent is the unpatented commercial variety Hydrangea serrata ‘Maiko’ The pollen parent is unknown, as it was an open pollination breeding program. The new variety was identified as a potentially interesting selection June of 2007, at a greenhouse in Grand Haven, Mich.

Asexual reproduction of the new cultivar ‘SMNSNFD’ by softwood cuttings was first performed during the June of 2007, at commercial greenhouse in Grand Haven, Mich. Subsequent propagation has shown that the unique features of this cultivar are stable and reproduced true to type in 5 successive generations.

### SUMMARY OF THE INVENTION

The cultivar ‘SMNMAKTSR’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SMNMAKTSR’ These characteristics in combination distinguish ‘SMNMAKTSR’ as a new and distinct Hydrangea cultivar:
1. Compact growth habit.
2. Hardy, frost resistant flower buds.
3. Re-blooming plants.
4. Large lace cap flowers.
5. Single and double florets, bright magenta/pink with yellow/green centers on double florets.

### PARENT COMPARISON

Plants of the new cultivar ‘SMNMAKTSR’ are similar to plants of the seed parent, Hydrangea serrata ‘Maiko’ in most horticultural characteristics, however, plants of the new cultivar ‘SMNMAKTSR’ differ in the following:
1. Single and double florets. The seed parent has only single florets.
2. Magenta pink florets, with centers of double florets colored yellow and green. The seed parent has lighter lavender solid colored florets.
3. Larger blooms.

Pollen parent unknown as this is a product of open pollination.

### COMMERCIAL COMPARISON

‘SMNMAKTSR’ can be compared to the patented commercial variety Hydrangea serrata ‘MAK20’, U.S. Plant Pat. No. 24,820. These varieties are similar in most horticultural characteristics; however ‘SMNMAKTSR’ differs in the following:
1. Single and double florets. The comparator has mostly single florets.
2. Magenta pink florets, with centers of double florets colored yellow and green. The comparator has lighter pink solid colored florets.
3. Large lace cap flowers. The comparator has medium lace cap flowers.

### BRIEF DESCRIPTION OF THE PHOTOGRAPHS

The accompanying photograph in FIG. 1 illustrates in full color a typical plant of ‘SMNMAKTSR’ grown in a double layered poly greenhouse, in Grand Haven, Mich., in a commercial 3 gallon container. Age of the plant photographed is approximately 3 years.

FIG. 2 illustrates typical flowers. The photographs were taken using conventional techniques and although colors may appear different from actual colors due to light reflectance it is as accurate as possible by conventional photographic techniques.

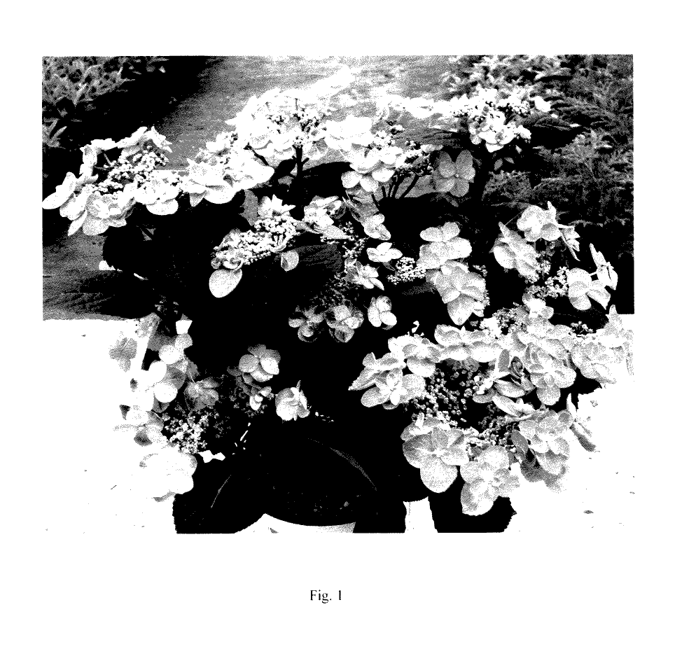

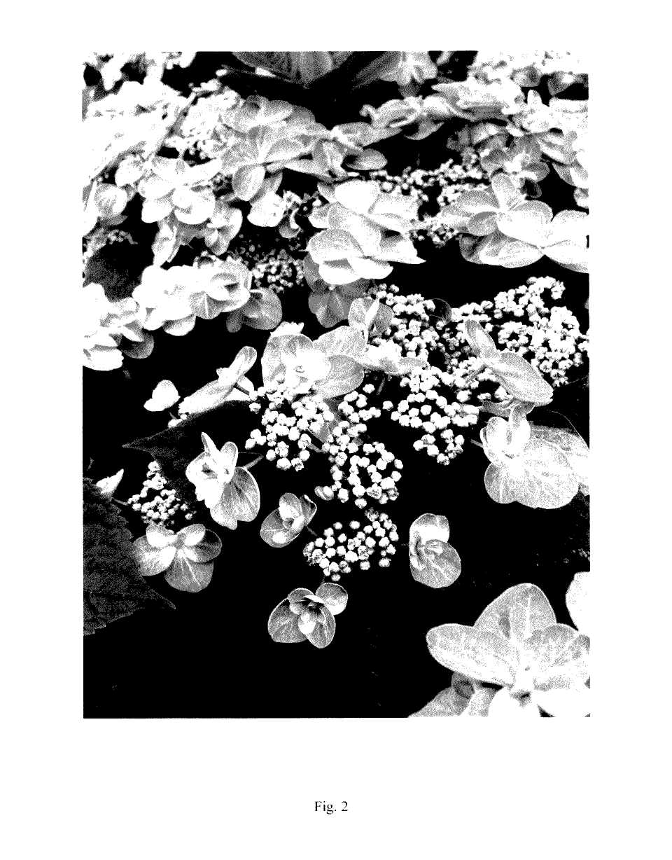

### DETAILED BOTANICAL DESCRIPTION

In the following description, color references are made to The 1996 Royal Horticultural Society Colour Chart except where general terms of ordinary dictionary significance are used. The following observations and measurements describe ‘SMNMAKTSR’ plants grown in a double layered poly greenhouse, in Grand Haven, Mich. The growing temperature ranged from approximately 18° C. to 27° C. daytime and 5° to 10° C. at night. Measurements and numerical values represent averages of typical plant types. Measurements were taken during April, 2015.
Botanical classification: Hydrangea hybrid ‘SMNMAKTSR.’.
Propagation:
   Time to initiate roots.—Approximately 10 days at approximately 20 ° C.
   Time to produce a rooted young plants.—Approximately 30 days at approximately 20 ° C.
   Root description.—Medium to thick, white, free to moderate root branching, both fibrous and fleshy.
   Propagation method.—Softwood cuttings.
Plant:
   Age of plant described.—3 years.
   Pot size.—3 gallon.
   Growth habit.—Flowering perennial shrub, mounded in form.
   Overall plant shape.—Upright, Rounded.
   Height.—Approximately 40 cm.
   Plant spread.—Approximately 70 cm.
   Growth rate.—Strong.
   Plant vigor.—Strong.
   Branching characteristics.—Good, lots of basal branches, pinching required.
   Length of lateral branches.—Approximately 40 cm.
   Number of lateral branches.—Approximately 20.
   Diameter of lateral branches.—0.6-0.8 cm.
   Lateral branch shape.—Round.
   Lateral branch strength.—Strong.
   Internode length.—Average range 4.5-7 cm.
   Branch color.—Immature: Near RHS Green 143C Mature: Near RHS Yellow-green 144B.
   Branch aspect.—Upright, outward.
   Branch pubescence.—None.
   Other stem or plant characteristics.—Oblong black lenticels.
Foliage:
   Leaf.—Arrangement: Opposite. Average Length: Approximately 11 cm. Average Width: Approximately 7 cm Shape of blade: Ovate-lanceolate Apex: Acuminate Base: Obtuse Margin: Serrulate Texture and appearance of top surface: Smooth, glabrous Texture and appearance of bottom surface: Slightly pubescent Color: Young foliage upper side: Near RHS Green 141A Young foliage under side: Near RHS Green 141B Mature foliage upper side: Near RHS Green 135A near stem, fading to Greyed-purple 187A towards margin. Mature foliage under side: Near RHS Green 138A Venation: Type: Pinnate Venation color upper side: Near RHS Greyed-orange 177B Venation color under side: Near Yellow-Green 145B.
   Petiole— Average Length: 1-1.5 cm Diameter: 0.5 cm Color: Upper side: Near RHS Greyed-orange 177B to Greyed-orange 166A nearest the stem. Under side: Near RHA Greyed-orange 177B Texture upper side: Slightly pubescent, ridged Texture under side: Smooth.
Flower:
   Bloom period.—Natural Season: Early Summer through Fall.
   Inflorescence.—Type: Lace cap Quantity of flowers per inflorescence: Fertile flowers, 250 approximately. Sterile Flowers, 10 approximately. Persistent or self-cleaning: Persistent. Fragrance: No.
   Panicle.—Shape: Orbital Height: 5 cm Diameter: 9 cm.
   Sterile flower.—Bud shape: Orbicular Bud length: 3 mm Bud diameter: 3 mm Bud color: Near RHS Greyed-purple 186C Flower aspect: Upright Flower shape: Obovate Flower length: 3.5 cm Flower Diameter: 3.5 cm. Flower Depth: 1.5 cm. Other characteristics: Very short and somewhat fused.
   Sterile flower petals.—Length of petal: Approximately 3 mm Width of petal: Approximately 2 mm Apex: Acute Shape of petal: Ovate Petal margin: Entire Petal Arrangement: Whorled Petal Base: Acute Petal Number: 6 Petals Fused: No. Petal Texture: Smooth, upper and under side.
   Color.—Upper surface at first opening: Near RHS Red-purple 70D Upper surface at maturity: Near RHS Red-purple 72D Under surface at first opening: Near RHS Red-purple 70D Under surface at maturity: Near RHS Red-purple 72D.
   Sterile flower sepal.—Arrangement: Whorl Number: 8 Shape: Oblong Tip: Obovate Base: Truncate Margin: Entire Length: 1.5 cm Width: 1.75 cm Texture, Upper: Smooth Texture, Lower: Smooth Color: Upper surface at first opening: Near RHS Red-purple 63B Upper surface at maturity: Near RHS Red-purple 63A Under surface at first opening: Near RHS Red-purple 62C Under surface at maturity: Near RHS Red-purple 62B.
   Fertile flower.—Bud shape: Orbicular Bud length: 3 mm Bud diameter: 3 mm Bud color: Near RHS Red-purple 68D Flower aspect: Upright Flower shape: Orbital Flower length: 4 mm Flower diameter: 4 mm Flower depth: 4 mm.
   Fertile flower petals.—Length of petal: 2.5 mm Width of petal: 1 mm Apex: Acute Shape of petal: Ovate Petal margin: Slightly undulate Petal Arrangement: Whorled Petal Number: 6 Petal base: Obtuse Petals Fused: No. Petal Texture: Smooth, upper and under side.
   Color.—Upper surface at first opening: Near RHS Red-purple 73C Upper surface at maturity: Near RHS Red-purple 73C Under surface at first opening: Near RHS Red-purple 73C Under surface at maturity: Near RHS Red-purple 73C.
   Fertile flower pedicel.—Angle: Outward/upright Strength: Moderately strong, flexible. Length: 1 cm Width: 1.5 mm Texture: Slightly pubescent Color: Near RHS Greyed-red 181B.
   Fertile flower sepal.—Arrangement: Whorled Number: 6 Shape: Ovate Tip: Obtuse Base: Obtuse Margin: Entire Length: 1 mm Width: 1 mm Texture, Upper: Smooth Texture, Lower: Smooth Color: Upper surface at first opening: Near RHS Yellow-green 144A Upper surface at maturity: Near RHS Yellow-green 144A Under surface at first opening: Near RHS Yellow-green 144A Under surface at maturity: Near RHS Yellow-green 144A.
Reproductive organs:
   Sterile flower.—Androecium: Stamen number: 6 Filament length: 2 mm Filament color: Near RHS Yellow-green 145C Anther shape: Club-like Anther color: Near RHS Yellow-green 145B Anther length: 1 mm Pollen amount: Moderate Pollen color: Near RHS Yellow-green 145B Gynoecium: Pistil number: 3 Pistil length: 1 mm Stimga shape: Flat Stimga color: Near RHS Greyed-purple 186B Style length: less than 1 mm Style color: Near RHS Greyed-purple 186B Ovary color: No ovary.
   Fertile flower.—Androecium: Stamen number: 10 Filament length: 3 mm Filament color: Near RHS Red-purple 73A Anther shape: Club-like Anther length: 1 mm Anther color: Near RHS Greyed-green 196C Pollen amount: Moderate Pollen color: Near RHS Greyed-green 196C Gynoecium: Pistil number: 5-6 Pistil length: 3 mm Stimga shape: Club-like Stimga color: Near RHS Red-purple 73C Style length: 1.5 mm Style color: Near RHS Red-purple 73C Ovary color: Near RHS Yellow-green 145B.
Other characteristics:
   Disease resistance.—Neither resistance nor susceptibility to diseases or pests has been observed in this variety.
   Temperature tolerance.—−25° C. to 37° C.
   Fruit/seed production.—No fruits/seeds detected to date.
   Garden performance.—Good tolerance for rain and wind.

What is claimed is:

1. A new and distinct cultivar of Hydrangea plant named ‘SMNMAKTSR’ as herein illustrated and described.
